(19) United States
(12) Patent Application Publication
(10) Pub. No.: US 2004/0186381 A1
(43) Pub. Date: Sep. 23, 2004

(54) Volume flow rate with medical ultrasound imaging

(75) Inventors: Qinglin Ma, Bellevue, WA (US); Grazyna M. Palczewska, Bellevue, WA (US)

Correspondence Address: Siemens Corporation, Intellectual Property Department, 170 Wood Avenue South, Iselin, NJ, 08830, US

(73) Assignee: Siemens Medical Solutions USA, Inc.

(21) Appl. No.: 10/797,377

(22) Filed: Mar. 10, 2004

Related U.S. Application Data
(60) Provisional application No. 60/456,160, filed on Mar. 20, 2003.

(51) Int. Cl. A61B 8/06

(52) U.S. Cl. 600/454

(57) ABSTRACT

Systems and methods are described for the manufacture and use of a dual-purpose array for ultrasound imaging. In one configuration, the array is useful as an array for normal imaging. This array can be designed as a 1.x-D (1.0, 1.25, 1.5, 1.75, etc.) array. In another configuration, the array is useful as a square annular array. While the principle architecture envisioned is a square ring, a rectangular ring or other approximation to a circular ring can be used when more rows and more complicated interconnects are used. In particular, when two annular arrays of different geometry are enabled, the attenuation compensated volume flow meter (ACVF) uniform method for measuring volume flow rate can be applied at desired time in a cardiac cycle. The systems and methods provide advantages because the array may be used for normal imaging in other applications, and still enable volume flow rate measurements. It allows the estimation of the volume flow rate and its other derivatives as an integral part of daily clinical workflow.

### REFERENCE TO RELATED APPLICATIONS

The present patent document claims the benefit of the filing date under 35 U.S.C. §119(e) of Provisional U.S. Patent Application Serial No. 60/456,160, filed Mar. 20, 2003, which is hereby incorporated by reference.

### BACKGROUND

The embodiments generally relate to the field of ultrasound imaging. The embodiments more specifically relate to quantitative assessment of blood flow.

In medical ultrasound, different operating modes provide multiple types of information. B-mode provides visual images of the organ. B-mode images give tissue pathology, show movement and are also used as landmarks for placing a Doppler sample volume or biopsy needle. Color Doppler images show the location of blood vessels and the flow in a qualitative way. Quantitative Doppler imaging provides more precise information about blood flow at a Doppler sample volume. However, these different modes of operation have different and sometimes conflicting requirement for beamformation. For example, B-mode beams may be oriented perpendicular to a boundary to best present the tissue and organ boundary. Doppler flow beams may be oriented in parallel with the vessel to best present flow information.

Cardiac volume flow rate, stroke volume and regurgitate fraction are some quantitative parameters for assessing the performance of a human or animal heart. Volume flow rate is invasively determined with thermal dilution. This invasive technique can be dangerous to the patient. Medical ultrasound provides a non-invasive technique to determine volume flow, a vessel cross sectional area times the spatial mean velocity (i.e., average velocity in the vessel cross sectional area). The existing method is to use the 2D B-mode or Doppler flow information to obtain the vessel diameter. Quantitative Doppler mode (i.e., spectral Doppler) information is used to obtain a representative spatial mean flow velocity. An assumption of a circular flow lumen is made to calculate the volume flow rate from the representative sample. This assumption may introduce an error, making reliable and accurate determination of volume flow difficult.

Other ultrasound methods have been used. For example, a uniform sensitivity approach (e.g., attenuation compensated volume flow) may achieve more accurate volume flow rate estimation. Attenuation compensated volume flow is disclosed in U.S. Pat. No. 5,085,220, the disclosure of which is incorporated herein by reference. Acoustic energy is used to measure volume flow by insonifying the vessel of interest with both wide and narrow beams from an annular array. Though these approaches may work well in the lab using a flow phantom, the device is difficult to position and in vivo reproducibility is poor. Breathing and heart beat movement can affect the aortic position and shape, resulting in inaccurate positioning of the acoustic beams.

### BRIEF SUMMARY

By way of introduction, the preferred embodiments described below include a method and systems for measuring a volume flow parameter with ultrasound. Ultrasound imaging is combined with uniform sensitivity measurement of volume flow. A more reliable and accurate cardiac output flow rate can be determined as part of two-dimensional imaging workflow. In order to add minimal extra work to a cardiac examination procedure, the volume flow rate estimation is performed with the same imaging transducer. The imaging mode is used to overcome the positioning difficulties of a blind Doppler device. The imaging capabilities of a normal imaging machine plus the volume flow information obtained using an annular array are provided.

In a first aspect, a method is provided for measuring a volume flow parameter with ultrasound. The volume flow parameter is measured as a function of acoustic energy transmitted from an annular configuration of elements of a transducer array. Two or three-dimensional ultrasound imaging is also performed with the transducer array.

In a second aspect, a system is provided for measuring a volume flow parameter with ultrasound. A transducer array has a plurality of elements. A processor is operable to calculate the volume flow parameter as a function of acoustic energy received with an annular configuration of elements of the transducer array. A display is operable to display the volume flow parameter and a two-dimensional image responsive to acoustic energy received with the transducer array.

In a third aspect, a transducer array is provided for both measuring a volume flow parameter and imaging with ultrasound. The array includes several rows of elements. A kerf separates one row of elements from another row of elements. The kerf may extend less than an azimuth length of the transducer array.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a graphic representation of transmit and receiver profiles and associated far field patterns in one embodiment;

FIG. 2 illustrates one embodiment of a square annular array;

FIG. 3 is a graphic representation of one embodiment of a spatial relationship for transmitting a uniform acoustic field;

FIG. 4 is a block diagram of one embodiment of a processing architecture;

FIG. 5 is a graphic representation of uniform and narrow beam insonification of a vessel;

FIG. 6 is a cross-sectional view of an elevation aperture of two transducer arrays;

FIG. 7 is a top view of a transducer array in a first embodiment;

FIG. 8 is a top view of a transducer array in a second embodiment;

FIG. 9 is a top view of a transducer array in a third embodiment; and

FIG. 10 is a top view of transducer array of FIG. 7 with an overlaid flex circuit pattern.

### DETAILED DESCRIPTION OF THE DRAWINGS AND PRESENTLY PREFERRED EMBODIMENTS

A method is provided for measuring a volume flow parameter with ultrasound. Both volume flow measurement and ultrasound imaging are provided on a same system, including a same transducer array. For uniform insonification, the elements of the transducer array are configured as annular array elements. For ultrasound imaging, the elements of the transducer array are used as 1D, 1.25D, 1.5D, 1.75D or 2D arrays. For example, the diagnostic medical ultrasound transducer array includes elements arranged in two, three or more physical rows. The elements in each row are connected to a beamformer for normal imaging. Normal imaging may include B-mode, M-mode, power, color Doppler, Doppler, Tissue Doppler, harmonic, contrast agent and/or other now known or later developed imaging. For volume flow measurements, the same or a sub-set of the same elements are connected to a beamformer as an annular array.

Beamformer operation, switches, interconnects, multiplex switches or other circuits are adapted to allow the choice of the different array configurations. The circuits may be located in the probe (including the handle) or in the ultrasound imaging system. For some of the embodiments, a second subset of the elements is used as a different annular array.

In order to provide both imaging and volume flow estimation with a same imaging system and same transducer array, the imaging system and transducer array are designed to allow both operations sequentially. The imaging system and transducer array embodiments are discussed below. The imaging generates images in any of the modes discussed herein, such as two-dimensional B-mode or Doppler mode images. One or three dimensional representations or images may also be generated.

In one embodiment, two-dimensional ultrasound imaging is performed with the transducer array. For example, a transducer array with three, five or other numbers of elevation spaced rows of azimuth spaced elements is operated as a 1.5D array. The elements of the center row are used to transmit and receive in response to apodization and delays from independent beamformer channels. The elements of pairs of rows on opposite sides of the center row are electrically connected together as pairs of elements, and the connected pairs of elements are used to transmit and receive in response to a same apodization and delays from a same beamformer channel. Different apodization and delays are applied with independent beamformer channels for the different pairs of elements. Acoustic energy is focused scanned along the azimuth dimension in any of now known or later developed one, two or three-dimensional scan patterns. The acoustic energy for 1.5D array operation is elevationally focused with no or minimal elevation steering. Alternatively, multiple rows are operated as a 1.25D (no independent beamformer channels along the elevation dimension or across rows), 1.75D (independent beamformer channels for all elements for three or five rows) or 2D (independent beamformer channels with a large number of rows of elements). A single row of elements may be used as a 1D array.

Based at least in part on one or more of the generated images, the, transducer array is positioned relative to a vessel of interest. For example, a two-dimensional B-mode or Doppler mode image is used to position the transducer such that a center scan line passes through the cross section, longitudinal section or combination thereof of a vessel of interest. Once the transducer array is positioned, the volume flow parameter is measured as a function of acoustic energy transmitted from an annular configuration of elements of the same transducer array. For example, the annular configuration is used to transmit a wide far field pattern and receive a wide and a narrow far field pattern. The responsive echo signals are used to calculate volume flow or other volume flow parameter. By using uniform sensitivity of the vessel with an annual array having similar or the same elevation and azimuth beam patterns, the volume flow is calculated from data providing the velocity across the entire vessel, rather than assuming velocity from a representative sample.

Volume flow rate is mathematically represented as:

$$Q = {\int }_{S}^{} V \cdot ⅆ S = \overline{V} \cdot S \qquad ( 1 )$$

where the spatial mean velocity is given by:

$$\overline{V} = \frac{{\int }_{S}^{} V \cdot ⅆ S}{{\int }_{S}^{} ⅆ S} . \qquad ( 2 )$$

Other representations of volume flow may be used. If the acoustic field that insonifies the flow lumen is generally uniform, the mean velocity estimated is an instantaneous spatial mean velocity that is independent of the velocity flow profile.

In order to obtain volume flow rate, the flow lumen area is determined. Since the backscattered power from red blood cells is typically proportional to the number of cells within the sample volume, the power may be proportional to the projected area. The power is mathematically represented as:

$$\begin{matrix}P = \int P ( f ) ⅆ f = I ( z ) \cdot \frac{S}{cos ( θ )} \cdot ρ \cdot α ( z ) & ( 3 ) \\ where \text{:} & \\ I ( z ) is the transducer sensitivity at depth z ; & ( 3 ) \\ S is the projected flow lumen area ; & \\ θ is the angle between the beam and the flow direction ; & \\ α ( z ) is the attenuation at depth z ; & \\ ρ is the volumetric scattering coefficient of blood . & \end{matrix}$$

A wide uniform far field acoustic pattern is transmitted, and wide and narrow far field acoustic pattern are received from the annular configuration of elements to measure volume flow based on the equations above. The narrow received beam is used to obtain the absolute numeric value of the flow lumen by canceling out the unknown factors θ, ρand α(z). A beam that is wide enough to encompass the entire vessel cross-section, such as a beam that is 2-4 centimeters wide at the region of interest, is transmitted. In response to that same transmission, the same wide beam plus a narrow beam are received. The ratio of the received Doppler power from the wide (P_{w}) and the narrow beam (Pₙ) is proportional to the lumen area as mathematically represented by:

$$\begin{matrix}{P}_{w} = \int {P}_{w} ( f ) ⅆ f = {I}_{w} ( z ) \cdot \frac{{S}_{w}}{cos ( θ )} \cdot ρ \cdot α ( z ) \\ {P}_{N} = \int {P}_{N} ( f ) ⅆ f = {I}_{N} ( z ) \cdot ρ \cdot α ( z ) \\ \overline{V} = {\overline{V}}_{0} \cdot cos ( θ ) \\ Q = k ( z ) \cdot \overline{V} \cdot \frac{{P}_{w}}{{P}_{N}}\end{matrix} \qquad ( 4 )$$

where k(z) is a depth dependent constant whose value depends on the shape and acoustic intensity distribution of the narrow beam, P_{w} is the power associated with the wide beam, PN is the power associated with the narrow beam and V is the velocity associated with the wide beam. From equation (4), the volume flow rate, Q, is independent of the Doppler angle and flow profile. The constant k(z) can be obtained by calibrating the system and transducer using known vessel sizes at different depths.

The uniform or wide transmit and/or receive beam is designed to encompass a majority, most or all any vessel likely to be of interest. For example, the beam is wide enough to encompass typical ascending aortas (e.g., 2 to 4 cm in diameter at 6 cm in depth). The far field pattern is the Fourier transform of the driving function as illustrated by FIG. 1. FIG. 1 shows a cross section of an annular arrangement of elements of an array 12, an associated driving function 14 across the elements, and the resulting far field pattern 16. On the left half of FIG. 1, a single element or piston of the annular array results in a relatively narrow far field beam pattern. The annular element configuration on the right half of FIG. 1, different polarity and apodization are used for transmit and receive as a function of the different annular elements as shown by the driving function 14. The center element has a positive polarity with relatively large amplitude. The adjacent ring or surrounding annular element has a negative polarity with relatively moderate amplitude (apodization). The polarity continues to switch between positive and negative and the amplitude continues to relatively decrease for each annular element spaced further from the center. Other apodization and polarity driving functions may be used, such as with no or different polarity switches between elements or as with different elements having greater or lesser relative amplitudes to other elements.

To form an annular array from a transducer also used for 2D or 3D dimensional imaging, groups of elements are formed or configured into annular elements. For example, a first group of elements from three different rows of elements is used as a ring annular element. A different group of elements from a single row of elements is used as a center annular element within the ring annular element. Other groups of elements forming other ring elements may be used. A ring element includes circular, oblong, rectangular, square and combinations thereof. The center annular element may be formed from elements in one or more rows of the transducer array. The ring annular elements may be formed from elements in two, three, four, five or more rows of the transducer array.

Where each annular element corresponds to a plurality of elements operated in a same way or as part of a single annular element, transmit waveforms with the same polarity and apodization are provided to the group of elements. For different ring annular elements, transmit waveforms with the same polarity and apodization are provided to the elements making up the different annular element. The polarity and apodization is different across the annular elements. The transmit waveforms are applied at a substantially same time to generate a beam the desired far field pattern, such as a wide or uniform beam. The same polarity and apodization is used to form the receive wide beam, but different polarity and apodization are used for receiving the narrow beam. The transmit waveforms that are the same are provided from a single beamformer channel through switching or through multiple beamformer channels generating the same waveform.

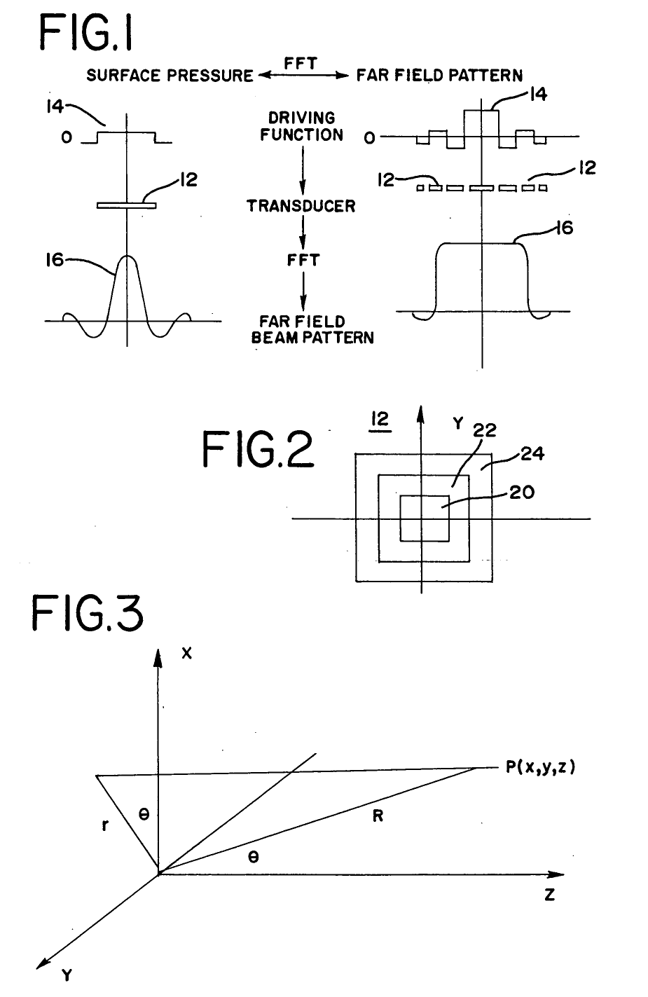

Any of various annular arrays may be used. A circular annular array is used in one embodiment for the uniform sensitivity method of measuring volume flow. Groups of elements are configured generally or substantially to form a circular annular pattern. As an alternative, a uniform annular square or rectangular array and associated elements are used. The square annular array is shown schematically by FIG. 2. A center element 20 is surrounded by two ring annular elements 22, 24. Additional or fewer ring elements 22, 24 may be used. One or more ring elements 22, 24 may be configured as two half or other number of sub-annular elements. The center element 20 may not be provided or used in some embodiments.

The driving function, D, for a square array is determined from the desired far field pattern, such as with the following function:

$$D ( θ , ϕ ) = \frac{sin ( \frac{π \cdot a}{λ} \cdot sin ( θ ) \cdot cos ( ϕ ) )}{\frac{π \cdot a}{λ} \cdot sin ( θ ) \cdot cos ( ϕ )} \cdot \frac{sin ( \frac{π \cdot a}{λ} \cdot sin ( θ ) \cdot sin ( ϕ ) )}{\frac{π \cdot a}{λ} \cdot sin ( θ ) \cdot sin ( ϕ )} \\ where \text{:} \\ x = R sin ( θ ) \cdot cos ( ϕ ) \\ y = R \cdot sin ( θ ) \cdot sin ( ϕ ) \\ z = R \cdot cos ( θ ) ≅ R \qquad ( 5 )$$

when θ is small. Solving for x, y, z coordinate frame:

$$D ( x , y ) = \frac{sin ( \frac{π \cdot a}{λ} \frac{x}{Z} )}{\frac{π \cdot a}{λ} \frac{x}{Z}} \cdot \frac{sin ( \frac{π \cdot a}{λ} \frac{y}{Z} )}{\frac{π \cdot a}{λ} \frac{y}{Z}} \qquad ( 6 )$$

where λ is the wavelength, α is the size of the center square along one side, Z is the focal depth, and θ and φ are the azimuth and elevation angle as shown in FIG. 3. FIG. 3 shows the spatial relationship of the angles within the x, y, and z coordinate system. The spatial angles and associated radii define the line along which the far field pattern is provided where the center of the center annular element is at 0, 0, 0 in x, y, z space. Solving for D in equation six provides the sinc function shown in the right half of FIG. 1 as the driving function 14. The zero crossings of the driving function define the width of the desired annular elements in cross section. The elements may deviate from this desired width. In some embodiments, the center annular element 20 is twice the width of the side or ring annular elements 22, 24 in one direction from the center of the annular array. The wide beam is formed by using all three (FIG. 2) or all four (FIG. 1) annular elements with apodization and alternating polarity determined by equation (6). The narrow beam is obtained by summing the signals from the most outer two (FIG. 1) or outer three (FIG. 2) annular elements. Other combinations of annular elements for forming the wide or narrow beam may be used.

Based on equations (4) above, the volume flow is calculated using the annular array and driving function above as a function of the velocity and power associated with the wide or uniform beam acoustic pattern and power associated with the narrow beam acoustic pattern. These power and velocity values are determined with a same imaging system also used for imaging.

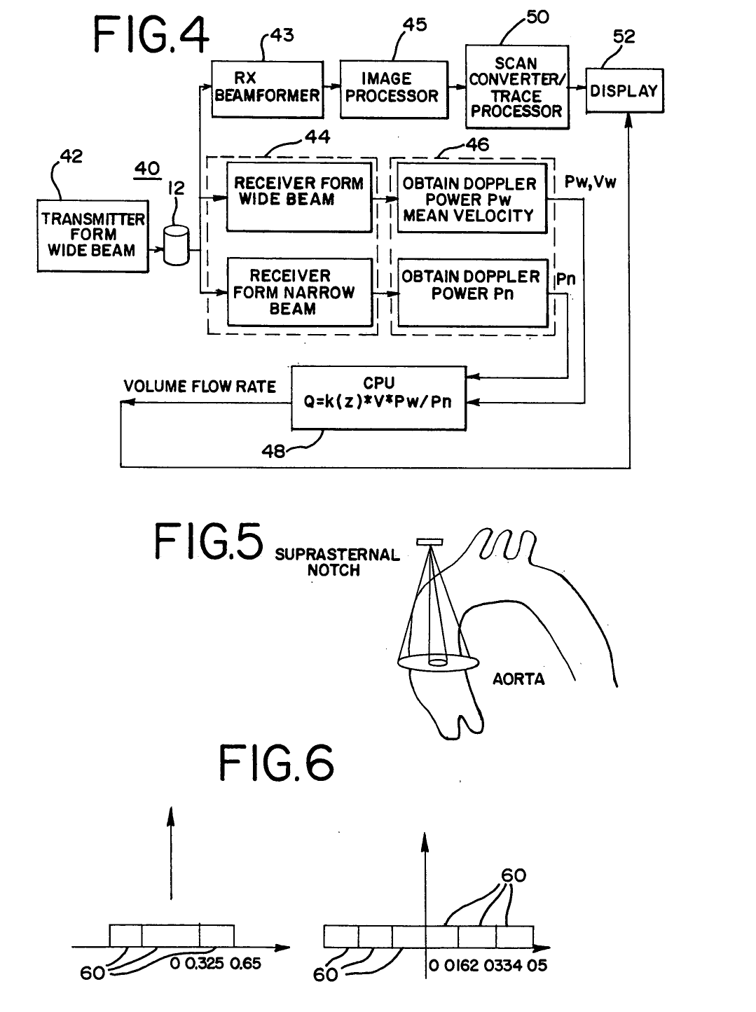

FIG. 4 shows one embodiment of a system 40 for measuring a volume flow parameter with ultrasound and imaging. The system 40 includes a transmitter 42, the transducer array 12, a receiver 44, a receive beamformer 43, a Doppler processor 46, an image processor 45, a processor 48, a scan converter 50 and a display 52. Additional, different or fewer components may be provided, such as a B-mode detector connected with the scan converter 50 for two or three dimensional B-mode imaging.

The transducer array 12 has a plurality of piezoelectric or capacitive membrane elements as discussed below. For example, a 1.5D array of elements is used. The elements of the array 12 are capable of interconnecting for imaging and interconnecting as annular array from all or a sub-set of elements. In one embodiment, a plurality of switches act as interconnects selecting between different configurations of elements. Alternatively, the operation of independent beamformer channels act as interconnects selecting between different configurations of elements. The annular configuration of elements or annular elements is operable to uniformly insonify a vessel with an aperture of similar azimuth and elevation sizes and associated beam patterns. For example, an annular configuration symmetrical about two dimensions is used as shown in FIG. 2. Oblong or rectangular annular arrays may also be used. The imaging configuration of elements is operable to insonify the vessel perpendicular to the scan lines or a C-section for imaging. Using the transducer array 12 for imaging, such as acquiring a three-dimensional volume, and also for determining volume flow may aid the positioning of the Doppler sample volume at the desired location.

The transmitter 42 is a transmit beamformer, waveform generator, amplifiers, delays, phase rotators, pulser or other now known or later developed transmitters for generating transmit waveforms for insonification. In one embodiment, the transmitter 42 is the transmit beamformer of the Siemens Medical Solutions, USA Sequoia™ ultrasound system or disclosed in U.S. Pat. No. 5,675,554, the disclosure of which is incorporated herein by reference. The transmitter 42 generates unipolar, bipolar or sinusoidal transmit waveforms for each of a plurality of channels. The transmitter 42 is operable to independently apodize and delay waveforms with selectable polarity for different channels at a same or similar time. For example, the transmitter is operable to simultaneously generate transmit waveforms with opposite polarity and different apodization for different annular elements of the annular configuration of elements of the transducer array 12. For the elements that are part of the same annular element, the same waveform is generated for each element by a respective channel. Alternatively, the same waveform is generated and provided to multiple elements configured within the same annular element.

The receiver 44 is a receive beamformer, a plurality of receive beamformers, amplifiers, delays, phase rotators, summer, summers, combinations thereof or other now known or later developed receivers for beamforming signals representing received acoustic echoes. The receiver 44 is operable to apodize with both positive and negative (inverted and non-inverted) weights. For example, the receiver 44 is operable to apply the polarity and amplitude weighting provided by the driving function for annular array operation. Relative delays and/or phase adjustments across the receive beamformer channels are implemented to focus the beams. For measuring volume flow, the receiver 44 is operable to simultaneously form two beams in response to a transmission. For example, a wide beam and a narrow beam are formed in response to the same transmission. The beams are formed from the same elements or different elements using different beamformer paths or storage of received signals and sequential processing with a same path. Receive beamformers for forming a plurality of beams at a same time using the same or some of the same elements are used in the Sequoia™ ultrasound system and shown in U.S. Pat. Nos. 5,555,534 and 5,685,308, the disclosures of which are incorporated herein by reference. The receiver 44 is also operable to receive beamform along scan lines for one, two or three-dimensional imaging.

The receive beamformer 43 is a same or separate component as the receiver 44. The receive beamformer 43 is used for imaging modes of operation. For example, a single beam is formed for M-mode or trace mode operation, or beams are scanning for two or three-dimensional imaging.

The Doppler processor 46 is a correlator, digital signal processor, processor, analog circuit, digital circuit, combinations thereof or any other now known or later developed Doppler processors. For example, the Doppler processor 46 includes both a processor and color-imaging path for estimating velocity, power or both for a plurality of spatial locations and a processor or spectral Doppler imaging path for determining signal spectral content for a particular location. The spectral content includes power and velocity as a function of time. Two Doppler paths are provided to obtain a first velocity and a first power associated with a uniform far field acoustic pattern as a function of the annular configuration and a second power associated with a narrow far field acoustic pattern as a function of the annular configuration. For volume flow calculation, the wide or uniform beam information is output by the receiver 44 to determine an associated power and velocity, and the narrow beam information is output by the receiver 44 to determine an associated power. In one embodiment, the color-imaging path is used to sequentially obtain Doppler power for both the narrow and wide beam information, and the spectral Doppler path is used to obtain the velocity from the wide beam information. Other distributions of processing may be used. For imaging, only one or both Doppler paths are used to generate a two or three-dimensional Doppler velocity or power image and/or a spectral Doppler image.

The image processor 45 is the same or different component as the Doppler processor 46. The image processor 45 is operable to detect and image process for any imaging mode of operation. For example, B-mode, Doppler mode, M-mode, spectral Doppler mode, harmonic mode, contract agent mode or other modes of detection are used to detect ultrasound data for imaging.

The processor 48 is a control processor, trace processor, general processor, digital signal processor, analog circuit, digital circuit, application specific integrated circuit or other now known or later developed device operable to calculate the volume flow parameter. The volume flow is calculated as a function of acoustic energy received with an annular configuration of elements of the transducer array. Volume flow is calculated as a function of the two beams received in response to transmission of the wide beam using annular elements. For example, the volume flow is calculated as a function of the uniform beam velocity, the uniform beam power and the narrow beam power.

The depth dependent coefficients k(z) is stored in a memory for use in calculating the volume flow. K(z) is determined by experimentation with similar systems and transducers or calibration of with each particular imaging system 40 and/or transducer 12. A table of k as a function of depth, z, is then extrapolated, interpolated or otherwise filled. Alternatively, the function k(z) is theoretically determined.

Other volume flow parameters may be calculated, such as cardiac volume flow rate, stroke volume and regurgitate fraction. The volume flow rate mode of operation is instantaneous upon user request, such as being initiated in response to a user depressing a button or positioning a marker on an image. Alternatively, the volume flow rate mode of operation is triggered by some cardiac event, such as peak systole. The output is the instantaneous spatial volume flow rate versus cardiac cycle and the Doppler power of the wide and narrow beam. Clinically relevant parameters, such as the stroke volume (e.g., cardio or organ transplant stroke volume), stroke length, forward and backward flow volume per cardiac cycle, the regurgitate fraction, instantaneous flow lumen and average flow lumen per cycle, may then calculated and output.

For imaging, the Doppler or B-mode information is output to the scan converter. The scan converter 50 converts from an acquisition format, such as a polar format, to a display format, such as a Cartesian format. The display 52 displays the volume flow parameter and/or an image responsive to acoustic energy received with the transducer array 12. For example, a two-dimensional B-mode and/or Doppler mode image is generated before, during and/or after measurement of the volume flow parameter. The image is responsive to at least one of the at least three rows of elements and to signals from the receiver focused as a function of apodization and delay along at least one row of elements of the transducer array. The volume flow information may be displayed as a graph or function of time, such as provided in a trace mode. The cardiac cycle may be tracked to synchronize the display of the volume flow information, allowing averaging over multiple heart cycles.

In addition to the imaging system 40, the transducer 12 is operable for use in both imaging and measuring volume flow. For example, the transducer array 12 comprises at least three rows 60 of elements 62 as shown in FIGS. 6-9.

1.5D transducer arrays 12 using multiple rows 60 are shown in U.S. Pat. No. 5,490,512, the disclosure of which is incorporated herein by reference.

FIG. 7 shows a top view of one embodiment of the transducer array 12. For cardiac imaging, the transducer array 12 is a 2 to 4 MHz phased array with a footprint of about 2 cm×1 cm. Any number of elements may be provided for use with a same or different number of beamformer channels. For example, 32 to 256 beamformer channels for use with 32 to more than 256 elements 62 may be used. In one embodiment, 64 to 96 elements 62 are used. In the embodiment of FIG. 7, there are 64 elements 62 in the imaging plane and 5 rows in the elevation plane. Each row 60 has 64 elements 62 spaced along the azimuth dimension, but a greater or fewer number of elements 62 may be provide in one, a sub-set or all of the rows 62. In this embodiment, the pitch of the elements 62 along the azimuth dimension is 0.3125 millimeters, but greater or less pitches may be used.

FIG. 6 shows two different transducer arrays 12 in cross-section along the elevation dimension. The elevation aperture has a center row 60 that is 0.3332 centimeters wide or 0.167 cm from the center. The width in elevation of each of the other rows 60 including width on each side of the center row 60 is also 0.3332 centimeters, such as extending out to 0.334 (first ring) and 0.5 (second ring) on each side of the center. With five rows, the total elevation aperture is about 1 centimeter. Other relative widths and number of rows may be used. In alternative embodiments, a two dimensional array is used.

A total of 64×3=192 electric channels are used for 1.5D. There are only three effective electrical rows 60 because elements 62 on the outer rows 60 are electrically tied to their respective element 62 on the opposite outer row 60, and elements 62 of the middle rows 60 are electrically tied to their respective element 62 on the opposite middle row 60. The electrical connection is hard wired or switched. For 128-channel imaging systems 40, the outer most 9 columns from both sides may be disabled.

The annular configuration is provided by groups of elements 62, such as one group of elements 62 from the at least three rows 60 of elements 62 arranged as a ring annular elements 64, 66. Another group of elements 62 uses a single row 60 to form the center annular element 68 within the ring annular elements 64, 66.

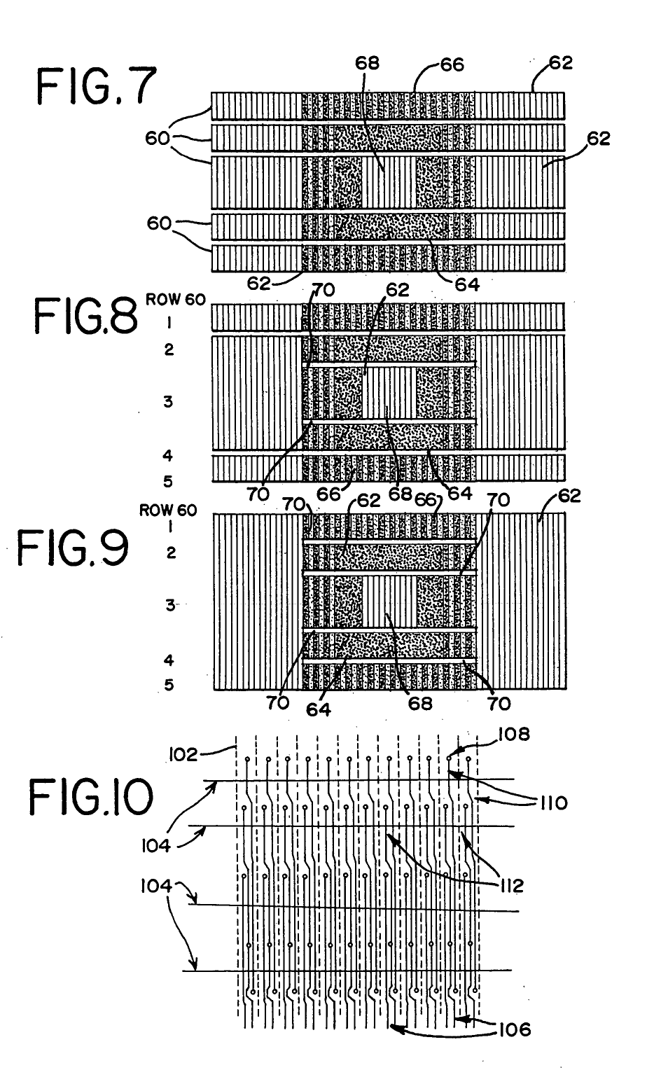

As shown in FIGS. 8 and 9, kerfs 70 may extend less than a full azimuth length of the array 12. Two rows 60 extend along a full length along an azimuth dimension as shown in FIG. 8. Another row 60 extends the full length when accounting for end elements 62, but also includes one or more kerfs 70 extending along the azimuth dimension less than the full length. The end elements 62 are used for imaging, but may not be used for the annular configuration. The kerfs 70 extend for a sufficient length to allow formation of the annular elements 64, 66, 62. The end elements 62 may have a same elevation width as a single row 60 or the extent of multiple rows 60. For example, the end elements 62 have an elevation with equal to two or more rows 60 plus the width of one or more kerfs 70. The interior rows 60 associated with the short kerf 70 extend a same length as the kerf 70 where the row 60 is characterized as ending at the first or inward most end element 62. The end elements extend from one or more rows 60 and kerfs 70 on each azimuth side of the array 12 to fill in the array for imaging. The kerfs or rows separation can be realized by: using the dicing saw, or laser or photolithographic technique on either the ceramic or flex circuit area connecting to the ceramic. The size of the rows and annular elements is selected to allow insonification through the suprastemal notch from the ascending aorta as shown by FIG. 5.

Electrical connection to each element is provided with a flex circuit with micro-vias, such as described in U.S. Pat. No. 5,617,865, the disclosure of which is incorporated herein by reference. This flex circuit can be used to provide internal connections between elements from rows 1 and 5, and between elements from rows 2 and 3, as shown on FIG. 10. FIG. 10 shows a flex circuit layout over the transducer elements of FIG. 7 for a 192 channel imaging system. The dashed lines 102 represent dicing cuts separating the elements in azimuth. The kerfs 104 separate the element rows in elevation. Flex circuit conductors 106 connect the elements to the system cables or channels. Plated micro vias 108 connect each conductor 106 to an element. The conductors 106 are routed on a side of the flex circuit spaced away from the transducer. The micro vias 108 then connect one or more elements to each system channel. By using the flex circuit to provide the internal connections between rows (e.g., conductors 110 connect elements on rows 1 and 5 and conductors 112 connect elements on rows 2 and 4), electronic switching in the transducer handle is avoided or the need for a large cable channel count is eliminated, reducing packaging size and cost.

Transducer array shown in FIG. 7 has 64 elements in the azimuth direction. Transducer arrays shown in FIGS. 8 and 9 have 96 elements in the azimuth direction. The total number of independent elements of the transducer shown in FIG. 7 is 5*64=320. Some kind of switching or multiplexing or internal connection method is needed for a system with less than 320 channels.

The embodiments of the transducer array 12 shown in FIGS. 7, 8 and 9 allow for matching a number of elements 62 to a number of beamformer channels, such as imaging systems 40 with 256-channel (FIG. 8) and 192-channel (FIGS. 7 and 9) without switching or a multiplexer. In FIG. 8, the outer twenty-four elements 62 from both sides of rows labeled 2, 3, and 4 are end elements, so are not diced through or extend across rows 2, 3 and 4. The total independent element count is 48 (from outer elements of row 1 and 5)+48 (from the outer elements of row 2, 3 and 4)+48*3 (from the inner elements of all rows)=240.

In FIG. 9, the outer twenty-four elements 62 from both sides of all rows are end elements 62, so are not diced through or extend across the entire elevation aperture. The total independent element count is 48 (from outer elements of all rows)+48*3 (from the inner elements of all rows)=192. Other embodiments with different numbers, arrangements and size of elements 62 may be used. A greater or lesser number of system channels may also be used, such as a 128-channel system. The transducer array 12 of FIG. 8 may be used by a 256-channel system as a 1D, a 1.5D or a 1.75D array for imaging.

While the invention has been described above by reference to various embodiments, it should be understood that many changes and modifications can be made without departing from the scope of the invention. For example, the individual components need not be formed in the disclosed shapes, or assembled in the disclosed configuration, but could be provided in virtually any shape, and assembled in virtually any configuration. Further, the individual components need not be fabricated from the disclosed materials, but could be fabricated from virtually any suitable materials. Furthermore, all the disclosed elements and features of each disclosed embodiment can be combined with, or substituted for, the disclosed elements and features of every other disclosed embodiment except where such elements or features are mutually exclusive.

It is therefore intended that the foregoing detailed description be regarded as illustrative rather than limiting, and that it be understood that it is the following claims, including all equivalents, that are intended to define the spirit and scope of this invention.

I (We) claim:

1. A method for measuring a volume flow parameter with ultrasound, the method comprising:
(a) measuring the volume flow parameter as a function of acoustic energy transmitted from an annular configuration of elements of a transducer array; and
(b) performing two-dimensional ultrasound imaging with the transducer array.

2. The method of claim 1 wherein (a) comprises:
(a1) transmitting a uniform far field acoustic pattern from the annular configuration of elements;
(a2) receiving a wide and a narrow far field acoustic pattern from the annular configuration of elements; and
(a3) calculating the volume flow parameter as a function of a first velocity and a first power associated with the uniform far field acoustic pattern and a second power associated with the narrow far field acoustic pattern.

3. The method of claim 1 wherein (b) comprises operating the transducer array as a 1.5D array.

4. The method of claim 1 wherein (b) comprises generating one of a B-mode and a Doppler mode image;
further comprising:
(c) positioning the transducer array relative to a vessel of interest based at least in part on the image.

5. The method of claim 1 wherein (a) comprises calculating volume flow with uniform sensitivity of the vessel.

6. The method of claim 1 wherein the transducer array comprises at least three rows of elements;
further comprising:
(c) using a first group of elements from the at least three rows of elements into a ring annular element for (a);
(d) using a second group of elements from at least one of the at least three rows of elements into a center annular element within the ring annular element for (a); and
(e) using at least one of the at least three rows of elements for (b).

7. The method of claim 6 further comprising:
(f) providing different transmit waveform polarity and apodization to different group of elements for (a) simultaneously; and
(g) focusing as a function of apodization and delay along the at least one row of elements for (b).

8. The method of claim 1 wherein (a) and (b) are performed with the transducer array having first and second rows extending a first length along an azimuth dimension and a third row extending the first length wherein the third row includes at least one kerf extending along the azimuth dimension less than the first length.

9. A system for measuring a volume flow parameter with ultrasound, the system comprising:
a transducer array having a plurality of elements;
a processor operable to calculate the volume flow parameter as a function of acoustic energy received with an annular configuration of elements of the transducer array; and
a display operable to display the volume flow parameter and a two-dimensional image responsive to acoustic energy received with the transducer array.

10. The system of claim 9 further comprising:
a first array interconnect capable of connecting the elements of the transducer array for two-dimensional imaging; and
a second array interconnect for connecting a first subset of the elements as an annular array for the annular configuration of elements.

11. The system of claim 9 further comprising:
a first Doppler path operable to obtain a first velocity and a first power associated with a uniform far field acoustic pattern as a function of the annular configuration; and
a second Doppler path operable to obtain a second power associated with a narrow far field acoustic pattern as a function of the annular configuration;
wherein the processor is operable to calculate the volume flow parameter as a function of the first velocity, the first power and the second power.

12. The system of claim 9 wherein the transducer array comprises a 1.5D array.

13. The system of claim 9 wherein the two-dimensional image comprises one of a B-mode and a Doppler mode image.

14. The system of claim 9 wherein the annular configuration of elements is operable to uniformly insonify a vessel with an aperture of similar azimuth and elevation sizes.

15. The system of claim 9 wherein the transducer array comprises at least three rows of elements, the annular configuration comprising a first group of elements from the at least three rows of elements arranged as a ring annular element and a second group of elements from at least one of the at least three rows of elements arranged as a center annular element within the ring annular element; and
wherein the two-dimensional image is responsive to at least one of the at least three rows of elements.

16. The system of claim 9 further comprising:
a transmitter operable to simultaneously generate transmit waveforms with opposite polarity and different apodization for different annular elements of the annular configuration; and
a receiver operable to simultaneously form two beams in response to a transmission;
wherein the two-dimensional image is responsive to first signals from the receiver focused as a function of apodization and delay along at least one row of elements of the transducer array and wherein the processor is operable to calculate volume flow as a function of the two beams received in response to transmission by the different annular elements.

17. The system of claim 9 wherein the transducer array comprises first and second rows extending a first length along an azimuth dimension and a third row extending the first length wherein the third row includes at least one kerf extending along the azimuth dimension less than the first length.

18. A transducer array for both measuring a volume flow parameter and imaging with ultrasound, the transducer comprising:
first and second rows of elements;
at least one kerf separating the first row of elements from the second row of elements, the at least one-kerf extending less than an azimuth length of the transducer array.

19. The transducer array of claim 18 further comprising at least third and fourth rows of elements, the first, second, third and fourth rows spaced along the elevation dimension in an order given by third, first, second and fourth, the third and fourth rows of elements extending the azimuth length of the transducer array, the first and second rows of elements extending a same azimuth length as the kerf, additional elements extending from the first row, second row and kerf from each azimuth side, the additional elements having an elevation width substantially equal to the elevation width of the first row, second row and kerf together.

20. The transducer array of claim 18 further comprising at least third, fourth and fifth rows, each of the rows extending the length of the kerf in the azimuth dimension; and
further comprising additional elements extending along the azimuth dimension from the first through fifth rows, an elevation width of the additional elements being greater than an elevation width of the elements of each of the first through fifth rows.
